(19) United States
(12) Patent Application Publication
(10) Pub. No.: US 2026/0216463 A1
(43) Pub. Date: Jul. 30, 2026

(54) Protective enclosure for an apparatus for delivering medical gas, such as NO

(71) Applicant: INOSYSTEMS, ANTONY (FR)

(72) Inventor: Nicolas TIRILLY, ANTONY (FR)

(21) Appl. No.: 19/416,505

(22) Filed: Dec. 11, 2025

(30) Foreign Application Priority Data
Jan. 24, 2025 (FR) 2500763

(51) Int. Cl. A61M 16/10; A61M 16/00

(52) U.S. Cl. CPC A61M 16/10; A61M 16/0003; A61M 2202/0275; A61M 2209/06

(57) ABSTRACT

The invention relates to a protective enclosure (1) for a gas delivery apparatus (100), typically an NO delivery apparatus, comprising a protective casing (2) which has a front face (10) and a bottom (50) and an internal compartment (3) sized and configured to accommodate a gas delivery apparatus (100). A first front cut-out (6) with a transparent pane (7) is made in the front face (10). A first cushioning element (4) is arranged on the bottom (50), inside the internal compartment (3), and a second cushioning element (5) is arranged on the front face (10) and protrudes away from said front face (10) of the protective casing (2).

### CROSS-REFERENCE TO RELATED APPLICATIONS

This patent application claims priority to French Application No. 2500763, filed January 24, 2025 and the entire contents of which are incorporated herein by reference.

### BACKGROUND

The invention relates to a protective enclosure intended to protect an apparatus for delivering a medical gas intended for a person, i.e. a patient, typically an NO delivery apparatus or device, in particular while the latter is being transported and used.

In the course of their medical treatment, some people, referred to as patients, receive a therapeutic gas that is administered to them by means of a gas administration apparatus or unit which feeds a patient circuit supplying the therapeutic gas to a respiratory interface, such as a respiratory mask, a tracheal intubation tube or the like, for administering therapeutic gas to the patients' airways.

Such is the case with inhaled nitrogen monoxide gas or "NO", in particular a combined mixture of NO/N₂/O₂ gases obtained by injection of a mixture of NO/N₂ gases typically containing between 100 and 2000 ppmv of NO (remainder N₂) into a stream of air or a mixture of O₂/N₂ containing at least 20 vol.% O₂ coming from a medical ventilator and conveyed by the patient circuit, such as a flexible pipe or the like.

The combined mixture of NO/N₂/O₂, in particular the NO it contains (e.g. dose of < 80 ppmv), makes it possible to dilate the blood vessels and increase oxygenation while improving the exchanges of gas in the patient.

It is therefore used to treat persistent pulmonary hypertension of the newborn (PPHN), acute respiratory distress syndrome (ARDS) in adults, or pulmonary hypertension in heart surgery, as disclosed in particular by EP-A-560928, EP-A-1516639 or US-A-10,201,564.

To inject the mixture of NO/N₂ gases into the patient circuit and produce the combined mixture of NO/N₂/O₂ gases, use is typically made of an NO delivery apparatus, as described for example in EP3906955, EP4209243 or EP4241812.

As recalled by EP4410345, certain apparatuses for supplying, i.e. delivering, gases such as NO must be able to be transported and used at an intervention site by rescue teams (emergency services, fire-fighters, etc.) and/or in emergency vehicles, such as an ambulance, an aircraft, a helicopter or the like.

However, when being transported or handled or the like, such a gas supply apparatus often experiences impacts, soiling, being dropped or other difficult weather or environmental conditions (for example rain, snow, wind, mud, etc.) that are likely to soil it, damage it and/or degrade it to the extent it becomes unusable, thereby giving rise to an obvious problem of safety for the patient to whom the therapeutic gas can then no longer be given.

Further bags, enclosures or the like for storing or transporting medical devices or apparatuses are disclosed in CN221654587, DE202010016037, DE202011005093, EP3791911 and DE102008064480.

One problem addressed by the invention is that of being able to avoid or reduce the risk of soiling and/or degradation of a gas supply apparatus, typically an NO delivery apparatus, in particular when it is used on the ground, i.e. at an intervention site (i.e. outside of a hospital) by a rescue team (emergency services, fire-fighters, etc.) and/or in an emergency vehicle (for example ambulance, aircraft, helicopter, etc.).

In addition, the gas supply apparatus must be able to continue to be used normally, in spite of these difficult use conditions, in particular the user must be able to access the display screen of the apparatus, any setting or selection buttons, the pneumatic and/or electrical connectors used for various couplings or connections, etc., normally.

### SUMMARY

A solution of the invention relates to a protective enclosure for an apparatus for delivering medical gas to a person, i.e. a patient, typically an NO delivery apparatus, comprising a protective casing which has a front face and a bottom, and an internal compartment sized and configured to accommodate a gas delivery apparatus, typically an NO delivery apparatus.

In addition, the protective enclosure, in particular the protective casing, comprises:

a first front cut-out, also referred to as front window, made in the front face of the protective casing, and a transparent polymer glass arranged in said first cut-out,

a first cushioning element is arranged on the bottom, inside the internal compartment, and

a second cushioning element is arranged on the front face and protrudes away from said front face of the protective casing, that is to say it protrudes outwards.

Depending on the embodiment in question, the protective enclosure of the invention may comprise one or more of the following features:

the transparent pane is resilient, i.e. flexible.

the protective casing forms a peripheral protective shell or structure inside which the apparatus is arranged, i.e. accommodated.

when an apparatus is accommodated in the protective casing, it rests on the first cushioning element arranged on the bottom.

the protective casing is made of a fabric comprising synthetic textile fibres, preferably polyester fibres.

the synthetic textile fibres are preferably coated with a protective substance (i.e. coated fabric), preferably a polymer, for example polyvinyl chloride or PVC.

the protective casing has a front face, a back face, a right-hand side, a left-hand side and a bottom, which are connected to one another, i.e. secured to one another.

the protective casing comprises or is made of a fabric connected to, i.e. reinforced by, at least one reinforcing plate, for example polymer reinforcing plate, typically a rigid plate.

the protective plate is arranged in the back face.

the protective casing further comprises a top which is made of synthetic textile fibres and protects the top portion of the product, while still allowing access to the carry handle.

the apparatus comprises a carry handle and the top of the protective casing is positioned underneath the handle, that is to say between the handle and the shell of the apparatus surmounted by said carry handle.

the top provides leaktight protection for the upper part of the apparatus.

the protective casing has a width (L) of between 35 and 40 cm, typically between approximately 28 cm and 33 cm, a depth (P) of between 8 and 20 cm, typically between approximately 10 cm and 18 cm, and a height (H) of between 18 and 40 cm, typically between approximately 20 cm and 34 cm.

the front face, the back face, the right-hand side, the left-hand side and the bottom, and preferably the top, comprise walls, typically a front wall, a back wall, a right-hand wall, a left-hand wall and a bottom wall, and preferably a top wall.

the internal compartment is delimited by said walls.

the front face, the back face, the right-hand side and the left-hand side are secured to the bottom and project upwards above the bottom.

the front face and the back face are connected to one another via the right-hand side and the left-hand side.

the front face and the back face are arranged parallel or substantially parallel to one another, that is to say they face one another in parallel. according to another embodiment, the front face and the back face are arranged such that one is slightly inclined with respect to the other, that is to say they face one another without being parallel.

the right-hand side and the left-hand side are arranged in such a way as to face one another, i.e. parallel or not parallel to one another.

the top wall closes the internal compartment from above.

the top wall is resilient, i.e. flexible.

the top wall (or top) can pivot with respect to the front face, preferably along an edge region forming a hinge, typically situated at the top of the front face.

the first cushioning element covers most of the interior surface of the bottom, preferably all of the interior surface of the bottom.

the first cushioning element is trapezoidal or rectangular parallelepipedal, preferably trapezoidal.

the bottom is trapezoidal or rectangular parallelepipedal, preferably trapezoidal.

the first cushioning element has a length of between 10 cm and 15 cm, a width of between 22 cm and 31 cm and a thickness of less than 5 cm, preferably of the order of approximately 2 cm to 3 cm.

the second cushioning element has a height of between 5 cm and 10 cm, a width of between 10 cm and 15 cm and a thickness/depth of between 1 cm and 3 cm.

a third cushioning element is arranged on the front face and protrudes away from said front face of the protective casing.

the third cushioning element has a height of between 5 cm and 10 cm, a width of between 1 cm and 3 cm and a thickness/depth of between 1 cm and 3 cm.

according to one embodiment, the second cushioning element and the third cushioning element have an (approximately) equal height.

the second cushioning element and third cushioning element are positioned on the front face, between the bottom and the first front cut-out comprising the transparent pane.

a second front cut-out is made in the front face of the protective casing.

the second front cut-out is situated between the second and the third cushioning element.

the first, the second and/or the third cushioning element comprise(s) a cushioning foam, for example made of cellular rubber, preferably made of nitrile-butadiene rubber (NBR).

the top wall is for example made of a fabric made of synthetic textile fibres.

the top wall is configured to pivot between (at least) a position referred to as the "closed" position, in which the user does not have access to the interior of the compartment, and a position referred to as the "open" position, in which the user can access the interior of the internal compartment.

a closure system keeps the top wall in the "closed" position.

the closure system comprises one or more connectors or the like, preferably two connectors.

each connector comprises a female portion and a male portion of which one is inserted into the other so as to form a closure system of the male/female type.

the male portion of each connector is borne by the back face or wall of the protective casing and the female portion of each connector is borne by the top wall, or vice versa.

the connector(s), in particular the male and female portions of each connector(s), are arranged on one or more straps.

it comprises two connectors arranged on two mutually parallel straps spaced apart by a distance of approximately 15 cm to 25 cm.

the male portion of each connector is secured to or borne by a strap (or strap portion) secured to the wall of the back wall of the protective casing, i.e. of the enclosure.

the two straps bearing the male portions of the two connectors cooperate with two fastening zones situated on the surface of the back wall, typically elongate zones, such that they are retained there by fixation via an adjustable fastening system which has two hook-and-loop strips having textures that differ from but complement one another so as to obtain a removable connection or fastening when they are brought into contact with one another, typically a system of the Velcro® fastener type.

the two straps bearing the female portions of the two connectors are secured, i.e. fixed, to the top of the protective casing, i.e. of the enclosure.

the back face of the protective casing has a wide central opening or rear window providing access to the internal compartment, preferably the rear window is situated between the two straps.

the right-hand side of the protective casing comprises multiple small panels placed side by side, also referred to as "tiling" or "tiles", in particular arranged such that they partially overlap and thus make it possible to discharge run-off water and to prevent it from penetrating into the casing.

the small panels are attached to the enclosure via their right-hand and left-hand edges, in particular at the vertical edges on the right-hand side of the enclosure.

at least some of the upper and/or lower edges of the small panels are free, i.e. not fixed, such that they form slots or "vents" providing access to the interior of the internal compartment.

some of the small panels are made of a transparent flexible polymer material, that is to say they are glazed and transparent.

some of the small panels are made of an opaque flexible material, such as a woven fabric made of synthetic textile fibres.

the left-hand side of the protective casing has a lateral cut-out in which a transparent, preferably flexible, pane is arranged so as to form a left-hand glazed lateral window, allowing the user to see the interior of the enclosure.

the transparent lateral pane extends over most of the surface area of the left-hand side.

the transparent pane(s), which is/are preferably flexible, are made of a transparent polymer sheet, plate, fabric, canvas or the like, preferably made of PVC, for example a PVC of the stained glass type.

the transparent lateral pane has one or more small openings which are made through said transparent lateral pane and are for example circular, oval or the like.

all or some of the four peripheral faces or walls of the casing are pierced with or comprise, typically a few mm above the bottom (for example around 2 cm from the bottom), a row of holes or orifices in fluidic communication with the internal compartment for discharging any water located in said internal compartment. Conversely, if the holes are too far down, that is to say too close to the bottom, and the product is put down into a puddle for example, water will penetrate into the product, and this should be avoided because the water can degrade the ventilator situated inside the casing. However, a small amount of water can be tolerated at the bottom provided that it is far enough away from the ventilator that is to be protected.all or some of the four peripheral faces or walls of the casing are pierced with or comprise, typically a few mm above the bottom (for example around 2 cm from the bottom), a row of holes or orifices in fluidic communication with the internal compartment for discharging any water located in said internal compartment. Conversely, if the holes are too far down, that is to say too close to the bottom, and the product is put down into a puddle for example, water will penetrate into the product, and this should be avoided because the water can degrade the ventilator situated inside the casing. However, a small amount of water can be tolerated at the bottom provided that it is far enough away from the ventilator that is to be protected.

the second and third cushioning elements positioned on the front face are covered with woven fabric, in particular the same woven fabric as that of which the front face of the casing is made.

Furthermore, the invention also relates to a ventilation assembly comprising a gas delivery apparatus, typically an NO delivery apparatus, accommodated in the protective casing of a protective enclosure according to the invention, in particular the one described above.

In other words, the invention also relates to the use of a protective enclosure according to the invention, for protecting a gas delivery apparatus, typically an NO delivery apparatus, in particular the one described above.

### BRIEF DESCRIPTION OF VIEWS OF DRAWINGS

The invention will now be better understood from the following detailed description, provided by way of non-limiting illustration, with reference to the appended figures, in which:

FIG. 1 schematically shows one embodiment of a protective enclosure for an apparatus for delivering gas, in this case NO, according to the invention, in 3/4 view from the right-hand side.

FIG. 2 is a 3/4 view, from the left-hand side of the protective enclosure in FIG. 1.

FIG. 3 is a view of the front face of the protective enclosure in FIG. 1.

FIG. 4 is a view from the left-hand side of the protective enclosure in FIG. 1.

FIG. 5 is a view from the right-hand side of the protective enclosure in FIG. 1.

FIG. 6 is a view of the back face of the protective enclosure in FIG. 1.

FIG. 7 schematically shows an NO delivery apparatus that can be protected by the protective enclosure in FIG. 1.

### DETAILED DESCRIPTION

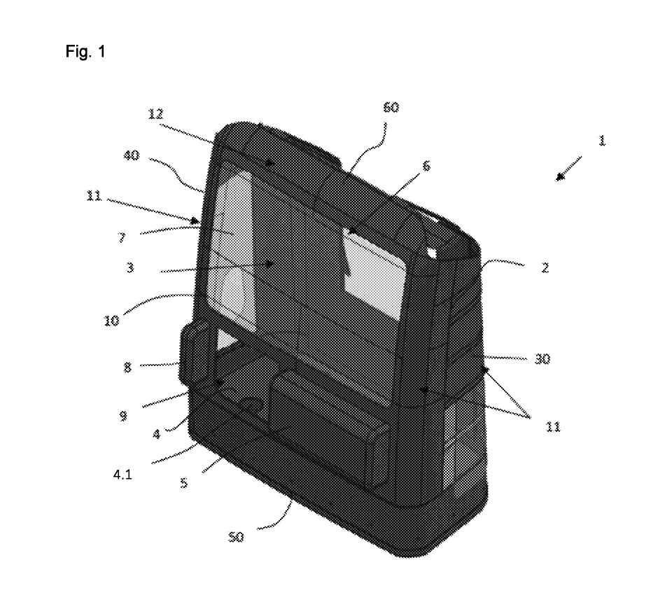

FIG. 1 to FIG. 6 provide various views of one embodiment of a protective chamber 1 according to the invention for accommodating a gas delivery apparatus, specifically in this case an NO delivery apparatus 100, as shown in FIG. 7.

The protective enclosure 1 according to the invention comprises a protective casing 2 comprising various walls 10-60 delimiting an internal compartment 3 sized and configured to accommodate the gas delivery apparatus 100 in FIG. 7, i.e. NO delivery apparatus. In this case, it is the NO delivery apparatus 100 sold under the trade name SoKinox®.

More specifically, the protective casing 2 comprises:

a front face 10 and a back face 20, which are arranged facing one another and separated from one another by the internal compartment 3,

a right-hand side 30 and a left-hand side 40, which are also arranged facing one another. The right-hand side 30 and the left-hand side 40 each join the front face 10 and the back face 20, in edge zones or regions 11, while being separated from one another by the internal compartment 3.

a bottom 50 surmounted by the walls forming the front face 10, the back face 20, and the right-hand side 30 and left-hand side 40, which walls project upwards from the bottom 50.

an upper wall or top wall 60 closing the internal compartment 3 from above. The top wall 50 is mounted pivotably with respect to the front face 10, along an edge region forming a hinge 12 (axis XX) situated at the top of the front face 10.

The top wall 60 is preferably flexible in order to allow it to pivot about the axis XX of the hinge 12 between (at least):

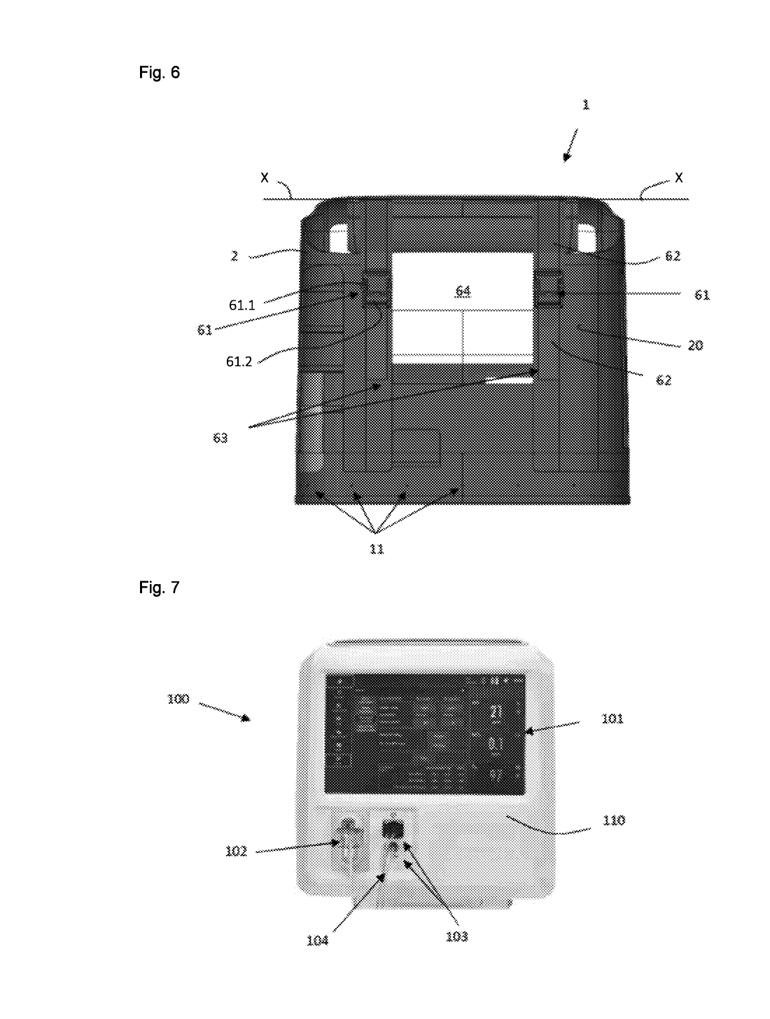

a position referred to as the "closed" position, illustrated in FIG. 6, in which the user does not have access to the interior of the compartment 3, and

a position referred to as the "open" position, in which the user can access the interior of the compartment 3 in order to insert the NO delivery apparatus 100 into it or extract the NO delivery apparatus therefrom.

Of course, while it is pivoting between these two positions, the top wall 60 can adopt other intermediate angular positions.

The top wall 60 may be held in the "closed" position by one or more suitable closure systems, for example two connectors 61 or the like, each comprising a female portion 61.1 borne by the top wall 60, into which is inserted a male portion 61.2 borne by the back face or wall 20, or vice versa, as illustrated in FIG. 6. The connectors 61 may be borne by straps 62 or the like, which are themselves secured to the top wall 60 and to the back wall 20.

The protective casing 2 is made for example of a woven fabric made of synthetic textile fibres, preferably made of polyester or polypropylene, which can be provided with a protective coat, preferably PVC, and can be connected to a reinforcing plate made of rigid plastic, preferably made of polypropylene or polycarbonate.

The reinforcing plate is situated in the back face, that is to say forms all or some of said back face.

It may be made of several walls or sub-units, i.e. panels or panel elements, which are joined to one another by adhesive bonding, thermowelding, stitching or the like.

In this case, the protective casing 2 is made of four peripheral panels or walls forming the front face 10, the back face 20 and the right-hand and left-hand sides, which are borne by the bottom 50 and secured thereto.

As illustrated in FIG. 1 to FIG. 3, the front face 10 has a first front cut-out 6, that is to say a front opening, which passes through the wall of the front face and which contains a transparent pane 7, i.e. a front pane, typically made of polymer, of low thickness, typically < 1 mm, preferably a resilient, i.e. flexible, transparent pane 7.

The front cut-out 10 and the transparent pane 7 form a glazed window, i.e. a front window, allowing the user to see the interior of the enclosure 1.

For example, the first front cut-out 6 may have a width of 24 cm to 26 cm and a height of 14 cm to 16 cm. The transparent pane 7 covers the entire surface area of the first front cut-out 6 and/or has substantially the same dimensions.

This front cut-out 6 forming a front opening is intended to be positioned facing the display screen 101 of the NO delivery apparatus 100, when it is accommodated in the internal compartment 3, so as to allow the user to view the displays (i.e. information, data, warnings, curves, values, doses, etc.) being shown on the display screen of the apparatus 100, while it is being used to treat a patient, and/or to make choices, selections or the like, by the user digitally pressing on the display screen 101 of the apparatus 100, via the transparent pane 7, if the display screen 101 is a touch-screen.

The internal compartment 3 comprises a first cushioning element 4 positioned on the bottom 50, in the interior of the internal compartment 3, on which the NO delivery apparatus 100 rests when it is accommodated in the internal compartment 3. This first cushioning element 4 may be removable.

The first cushioning element 4 closely follows the internal contours of the compartment 3 of the protective casing 2 and the internal surface of the bottom 50. It may for example be made of a plate or the like extending over all or some of the bottom 50 of the internal compartment 3, preferably over the entire bottom 50, on the inside.

This first cushioning element 4 may be made of a cushioning material such as a cushioning foam, for example made of cellular rubber of NBR type or the like, for absorbing impacts, vibrations or the like, in particular for providing cushioning in the event of being dropped on the ground, in particular impacts against the bottom 50, typically if the enclosure is dropped from a height of up to approximately 50 cm onto its bottom 50.

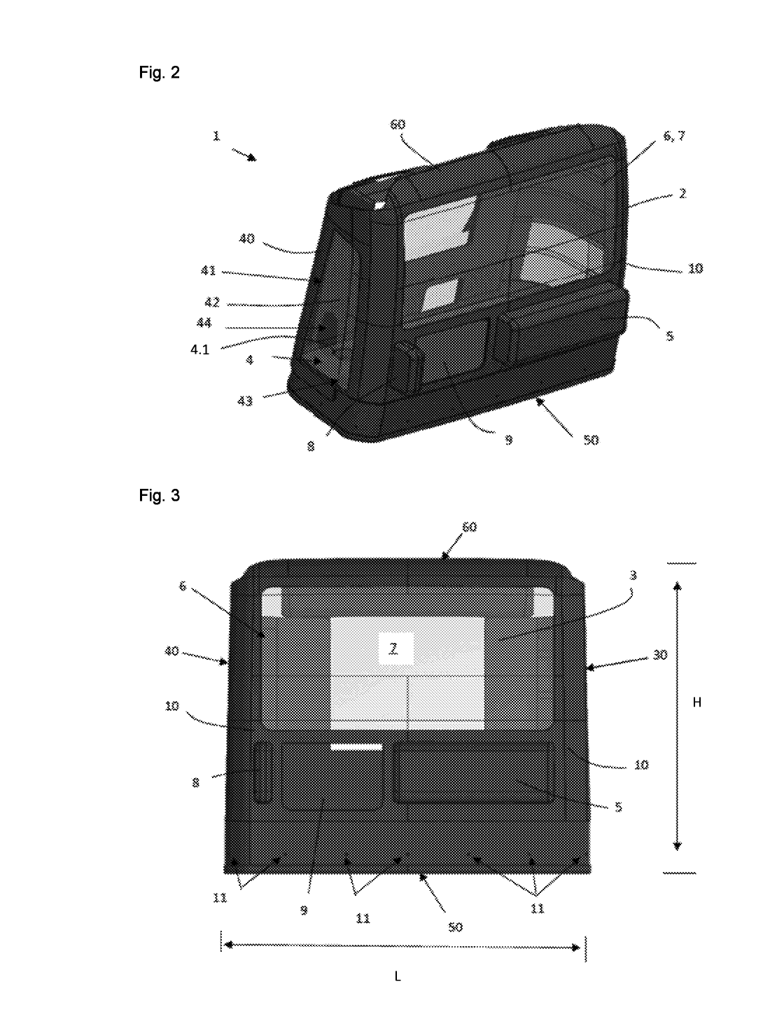

Advantageously, the first cushioning element 4 may comprise small receiving portions or recesses 4.1 intended to receive the feet of the apparatus 100 when it is positioned therein, for example four recesses 4.1 made in the upper surface of the first cushioning element, i.e. in the surface of the foam or the like, for receiving the four feet of the apparatus 100, as can be seen in FIG. 1 and FIG. 2.

Furthermore, the front face 10 further comprises a second cushioning element 5 arranged on the front face 10, the second cushioning element protruding away from said front face 10 of the protective casing 2, i.e. projecting outwards.

Advantageously, the front face 10 may comprise a third cushioning element 8, also arranged on the front face 10 and protruding away from said front face 10 of the protective casing 2, that is to say it too projects outwards.

The second and the third cushioning element 5, 8 are also intended to absorb or cushion impacts, vibrations or the like, in particular in the event of possibly being dropped onto the ground, thereby likely degrading the front face 110 and the screen 101 of the NO delivery apparatus located in the compartment 3.

The second and the third cushioning element 5, 8 may be made of the same cushioning material as the first cushioning element 4 or another material such as a polyethylene (PE) foam, an ethylene/vinyl acetate (EVA) foam or the like.

The second cushioning element 5 has a size greater than that of the third cushioning element 8, for example the second cushioning element 5 has a width of between 1 and 3 cm, a height of between 6 and 7 cm and a depth of between 2 cm and 3 cm, whereas the third cushioning element 8 has a width of between approximately 12 and 14 cm, a height of between approximately 6 and 7 cm, and a depth of between approximately 2 and 3 cm.

As can be seen in FIG. 3, the second and the third cushioning element 5, 8 are situated underneath the glazed window 6, 7, that is to say between the glazed window 6, 7 and the bottom 50 of the protective casing 2 of the enclosure 1.

Furthermore, a second front cut-out 9 is made in the wall of the front face 10 of the protective casing 2 as can be seen in FIG. 1 to FIG. 3. The second front cut-out is situated between the second and the third cushioning element 5, 8 and therefore also the glazed window 6, 7.

This second front cut-out 9 makes it possible to access the interior of the internal compartment 3, that is to say the apparatus situated in the internal compartment 3 of the protective casing 2, in particular elements situated underneath the screen 101 of the apparatus 100, such as a water trap device 103, connection ports 103 for connecting pipes or electric cables 104 or the like, as illustrated in FIG. 7.

The second and the third cushioning element 5, 8 also make it possible to protect the water trap device 103 and the connection ports 103 of the apparatus 100 in the event of being dropped onto its front face 10.

FIG. 1 and FIG. 5 show the right-hand side 30, i.e. the right-hand lateral wall, of the protective casing 2 of the protective chamber 1 of the invention.

As can be seen, the right-hand side 30 comprises multiple small panels 31 placed side by side, also referred to as "tiles", in particular arranged in this case one above another.

The small panels 31, i.e. their right-hand edges 31.1 and left-hand edges 31.2, are attached to the enclosure 2 at the vertical borders 32.1, 32.2, respectively, on the right-hand side 30 of the enclosure 2, as illustrated in FIG. 5.

By contrast, at least some of the upper and lower edges 33 of the small panels 31 are not fixed to one another such that they form slots 34 or "vents" providing access to the interior of the internal compartment 3. Cables or pipes can be passed through these slots 34.

Some small panels 31 may cover one another slightly, i.e. partially overlap, in the regions of their upper and lower edges 33, so as to improve the protection provided against intruding pollutants or bad weather, in particular against rain, snow or the like.

The small panels 31 may have various shapes, in particular trapezoidal, square, rectangular or other shapes. For example, they may have a width of between approximately 7 and 10 cm, and a height of between approximately 2 and 5 cm.

As can be seen in FIG. 5, the small panels 31 situated in the top portion 30A on the right-hand side 30, for example 2 or 3 panels 31, may be made of a woven and opaque material, for example a preferably elastic fabric made of polyester and elastodiene, typically an elastic strip.

Furthermore, the small panels 31 situated in the bottom portion 30B on the right-hand side 30, for example 2 or 3 panels 31, are preferably made of a transparent polymer material, preferably a flexible transparent polymer material, forming a small pane 35 similar to the transparent pane 7, i.e. the front pane.

These small paned 35 panels 31 allow the user to read information present on the apparatus 100 when the latter is arranged in the internal compartment 3, such as a product label or the like. Again, the slot 34 situated between two small paned 35 panels 31 provides access to the interior of the internal compartment 3 and allows a cable or pipe to pass through.

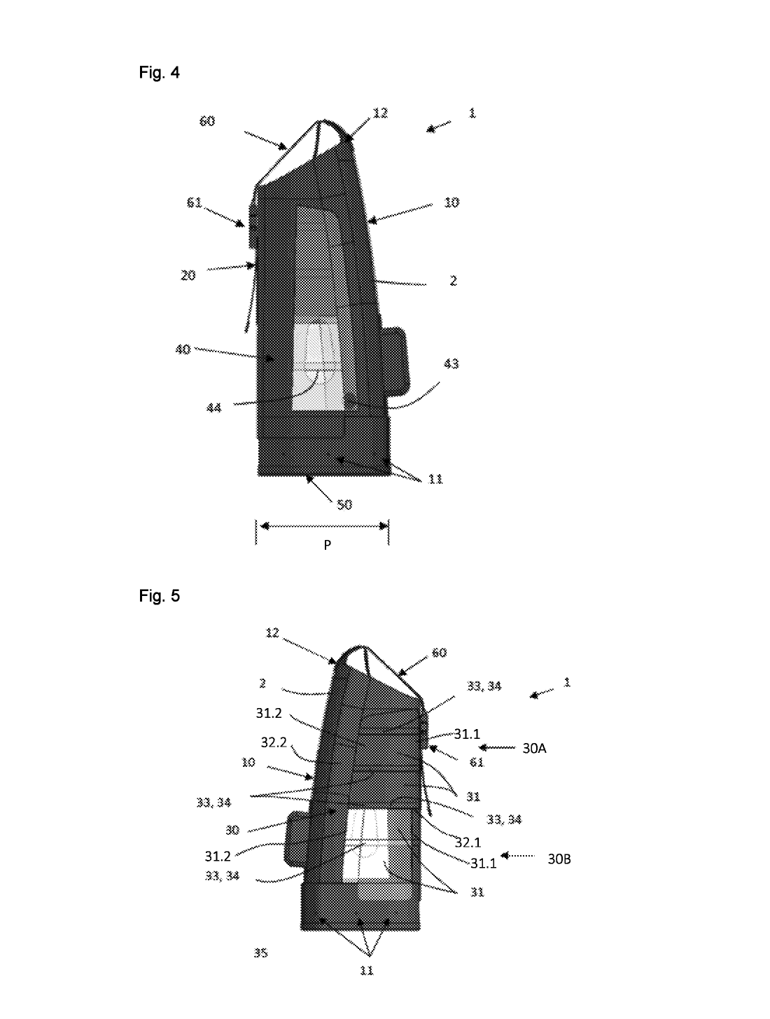

FIG. 2 and FIG. 4 show the left-hand side 40, i.e. the left-hand lateral wall, of the protective casing 2 of the protective chamber 1 of the invention.

As can be seen, the left-hand side 40 comprises, in a similar way to the front face 10, a lateral cut-out 41 which passes through the wall of the left-hand side and which also contains a transparent pane 42, preferably a flexible transparent pane, i.e. a lateral pane, typically made of polymer, of low thickness, typically < 1 mm. The lateral cut-out 41 and the transparent pane 42 again form a glazed window, i.e. a left-hand lateral window, allowing the user to see the interior of the enclosure 1.

This transparent lateral pane 42 extends over most of the surface area of the left-hand side 40. In this case, it has a substantially trapezoidal shape but could have another shape, for example a rectangular or other shape.

However, it should be noted that in this case, two small openings 43, 44, specifically in this case a circular opening 43 and a "pear"-shaped oblong opening 44, are made through the transparent lateral pane 42 and allow one or more cables to be passed through or a rotary setting-control knob to be accommodated for example.

Moreover, FIG. 6 shows the back face 20 of the protective casing 2 of the protective enclosure 1 according to the invention.

The back face 20 comprises, as already explained, the connectors 61 serving to hold the top wall 60 in the closed position.

With preference, the male portion 61.2 of the two connectors 61 are secured to straps 62 or the like, which are themselves secured to the wall of the back wall 20, as illustrated in FIG. 6. The two straps 62 are arranged parallel to one another and spaced apart by a distance of approximately 15 to 25 cm.

With preference, the two straps 62 cooperate with two fastening zones 63 situated on the surface of the back wall 20, typically elongate zones, i.e. vertically oriented zones, such that they are retained there by fixation via an adjustable fastening system which has two hook-and-loop strips having textures that differ from but complement one another so as to obtain a removable connection or fastening when they are brought into contact with one another, typically a fastening system with small hooks borne by one of the strips and with small loops borne by the other of the strips, which small hooks and small loops cooperate together to ensure retention, i.e. a system of the Velcro® fastener type.

The back face 20 is preferably more rigid than the front face 10 and the lateral panels 30, 40 and top panel 60. The back face 20 may be made of a more rigid material, such as a reinforcing plate made of rigid plastic, preferably polypropylene or polycarbonate, and/or comprise stiffeners such as material or foam portions of greater thickness.

Furthermore, it can be seen that the back face 20 comprises a wide central opening or rear window 64, situated between the two straps 62, which provides access to the back of the apparatus 100 when it is accommodated in the internal compartment 3.

Specifically, an NO delivery apparatus 100 conventionally comprises a back mount referred to as a Vesa® mount, used for mounting the apparatus 100 on a support.

The rear window 64 is therefore configured and sized to make it possible to use the Vesa® mount without having to remove the enclosure 1 protecting the apparatus 100, i.e. to be able to mount the apparatus 100 on a support while continuing to provide it with protection by means of the protective enclosure 1.

Therefore, the rear window 64 preferably has a rectangular, square or similar shape. Its dimensions are approximately 140 cm x 150 cm.

Furthermore, FIG. 1 to FIG. 6 show that the 4 sides 10-40 of the casing 2 forming its peripheral wall are pierced with or comprise at their base, that is to say just above the bottom 50, a row of holes or orifices 11 for discharging any water that might enter or be located in the internal compartment 3 of the casing 2, during use or rescue outdoors owing to poor weather, for example in the event of rain or snow.

More generally, the protective enclosure 1 may have a width L of between 23 and 33 cm, a depth P of between 10 and 19 cm, and a height H of between 27 and 34 cm, in order to be able to accommodate a gas delivery apparatus, typically an NO delivery apparatus 100.

In addition, the protective enclosure 1 is preferably lightweight in order to allow it to be easily transported, for example it may have a weight (mass) of less than 2 kg, preferably less than 1.5 kg, typically between approximately 0.7 kg and 1 kg.

The protective chamber 1 has the advantages of:

providing good protection for the apparatus 100 it contains against impacts, being dropped, vibrations or the like, or against bad weather, such as rain, snow, etc. and against soiling, such as dust, mud, etc.

being easy for the user, i.e. the medical personnel, to transport.

being easy to use.

not forcing the user to take out the apparatus 100 in order to be able to use it.

being solid and relatively insusceptible to tears or the like.

being in accordance with the relevant standards, in particular the standards IEC 60601-1-12:2014 and NF EN 1789:2021.

Lastly, FIG. 7 shows a gas, i.e. NO, delivery apparatus 100 that can be protected by the protective enclosure 1 in FIG. 1, that is to say a protective enclosure 1 according to the invention.

However, this example is non-limiting and the protective enclosure 1 according to the invention can be used to protect other gas, in particular NO, delivery apparatuses.

What is claimed is:

1. Protective enclosure (1) for a gas delivery apparatus (100) for delivering medical gas to a patient, typically an NO delivery apparatus, comprising a protective casing (2) which has a front face (10) and a bottom (50) and an internal compartment (3) sized and configured to accommodate a medical gas delivery apparatus (100), characterized in that:
a first front cut-out (6) is made in the front face (10) of the protective casing (2), and a transparent polymer pane (7) is arranged in said first cut-out (6),
a first cushioning element (4) is arranged on the bottom (50), inside the internal compartment (3), and
a second cushioning element (5) is arranged on the front face (10) and protrudes away from said front face (10) of the protective casing (2).

2. Enclosure according to claim 1, characterized in that the first cushioning element (4) covers most of the interior surface of the bottom (50), preferably all of the interior surface of the bottom (50).

3. Enclosure according to claim 1, characterized in that a third cushioning element (8) is arranged on the front face (10) and protrudes away from said front face (10) of the protective casing (2).

4. Enclosure according to claim 3, characterized in that the second cushioning element (5) and third cushioning element (8) are positioned on the front face (10), between the bottom (50) and the first front cut-out (6) comprising the transparent pane (7).

5. Enclosure according to claim 3, characterized in that a second front cut-out (9) is made in the front face (10) of the protective casing (2), said second front cut-out (9) being situated between the second and the third cushioning element (5, 8).

6. Enclosure according to claim 4, characterized in that the first, the second and/or the third cushioning element (4, 5, 8) comprise(s) a cushioning foam.

7. Enclosure according to claim 1, characterized in that the protective casing (2) comprises the front face (10), a back face (20), a right-hand side (30), a left-hand side (40) and the bottom (50), which are connected to one another, and a top (60) arranged pivotably (XX) with respect to the front face (10).

8. Enclosure according to claim 7, characterized in that all or some of the four faces (10-40) of the casing are pierced with or comprise a row of holes or orifices (11) in fluidic communication with the internal compartment (3).

9. Enclosure according to claim 1, characterized in that the protective casing (2) is made of a fabric comprising synthetic textile fibres, preferably polyester fibres.

10. Enclosure according to claim 9, characterized in that the synthetic textile fibres are coated with a protective substance, preferably polyvinyl chloride (PVC).

11. Ventilation assembly (1, 100) comprising a medical gas delivery apparatus, typically an NO delivery apparatus (100), accommodated in the protective casing (2) of a protective enclosure (1) according to claim 1.

12. Use of a protective enclosure (1) according to claim 1, for protecting a medical gas delivery apparatus, typically an NO delivery apparatus (100).
